(19) United States
(12) Patent Application Publication
(10) Pub. No.: US 2021/0388023 A1
(43) Pub. Date: Dec. 16, 2021

(54) ANALYTICAL TOOL FOR CHARACTERIZATION OF LIPID NANOPARTICLES

(71) Applicant: Merck Sharp & Dohme Corp., Rahway, NJ (US)

(72) Inventors: Sha Ha, Blue Bell, PA (US); John W. Loughney, Bethlehem, PA (US); Kevin A. Minsker, Lansdale, PA (US); Richard R. Rustandi, Lansdale, PA (US)

(73) Assignee: Merck Sharp & Dohme Corp., Rahway, NJ (US)

(21) Appl. No.: 17/289,095

(22) PCT Filed: Nov. 1, 2019
(86) PCT No.: PCT/US2019/059307

§ 371 Date: Apr. 27, 2021

Related U.S. Application Data
(60) Provisional application No. 62/756,302, filed on Nov. 6, 2018.

(51) Int. Cl. C07K 1/28; A61K 9/127

(52) U.S. Cl. CPC C07K 1/28; A61K 9/1272

(57) ABSTRACT

Described herein are methods of separating lipid nanoparticles (LNPs) according to their isoelectic points, the methods comprising applying a separating voltage to a separation matrix comprising carrier ampholytes and the LNPs for a sufficient time to separate the LNPs according to their isoelectic points.

### CROSS REFERENCE TO RELATED APPLICATIONS

This application claims the benefit of U.S. Provisional Application No. 62/756,302, filed Nov. 6, 2018, the content of which is hereby incorporated by reference in its entirety.

### BACKGROUND OF THE INVENTION

Lipid nanoparticles (LNP) have been employed for drug delivery in small molecules, siRNA, mRNA, and pDNA for both therapeutics and vaccines. Characterization of LNPs is challenging because they are heterogeneous mixtures of large complex particles. Many tools for particle size characterization, such as dynamic and static light scattering, have been applied as well as morphology analysis using electron microscopy. Capillary electrophoresis has been applied for the characterization of many different large particles such as liposomes, polymer, and viruses. However, there have been limited efforts to characterize the surface charge of LNPs and capillary isoelectric focusing has not been explored for this type of particle.

The inventors have developed an imaged capillary isoelectric focusing (icIEF) method to measure the surface charge (e.g., pI) of an LNP containing an ionizable lipid (e.g., a cationic lipid). The methods disclosed herein can distinguish the pI of LNPs manufactured with one or more different ionizable lipids for the purpose of confirming LNP identity in a manufacturing setting. Additionally, the methods are quantitative and stability-indicating making them suitable for both process and formulation development.

### SUMMARY OF THE INVENTION

The present invention relates to methods of separating lipid nanoparticles (LNPs) according to their isoelectic points, the methods comprising applying a separating voltage to a separation matrix comprising carrier ampholytes and the LNPs for a sufficient time to separate the LNPs according to their isoelectic points. The separation matrix may further comprise a stabilizer (e.g., glycerol or sucrose), methycellulose, pI markers, or any combination thereof. The methods can be performed in a capillary. The separation matrix can be imaged (e.g., by detecting UV absorbance at 280 nm) following separation to produce an electropherogram. The electropherogram can be compared to reference electropherograms, for example, to evaluate batch consistency, changes in LNP stability, or to identify or verify the identity of ionizable lipids (e.g., anionic or cationic lipids) in an LNP. The measured peak area in the electropherogram can be used to calculate lipid levels.

An aspect of the invention relates to an imaged capillary isoelectric focusing (icIEF) method for analyzing and/or characterizing LNPs formulated with or without an active ingredient. The icIEF charge based method measures the pI of LNPs. The observed signal is in proportion to the LNP diameter (size) and lipid concentration. The diameter for exemplary LNP's ranges from about 60 to about 150 nm, or 70 to about 140 nm, and lipid concentration ranges from about 7 to about 115 μg/mL total lipids.

An aspect of this invention relates to an imaged capillary isoelectric focusing (icIEF) to method for analyzing a composition comprising empty or encapsulated RNA or DNA constructs (e.g., mRNA, dsRNA, siRNA, and/or peptide LNPs). Another aspect of the invention relates to a method of determining the isoelectric point (pI) of the cationic lipid of LNPs. Still another aspect of the invention relates to a method of determining the isoelectric point (pI) of LNPs with or without, for example an oligonucleotide, RNA or DNA construct. Still another aspect of the invention relates to determining the stability of LNP compositions with or without active ingredients by employing capillary isoelectric focusing to determine the pI of the LNP. Another aspect of the invention relates to a method for identifying and/or separating mixtures of LNPs containing different cationic lipids by using capillary electric focusing to measure the pI of such cationic lipids. By subjecting LNPs to capillary isoelectric focusing, the LNP is separated based on its total charge. The total charge is largely dependent upon the cationic lipid used within the LNP.

An aspect of this invention is realized when the pI of the LNP is determined using an icIEF instrument (e.g., from Protein Simple). The method is robust, can be used for quantitation of LNP, and is capable of analyzing different LNPs containing different cationic lipid as a potential for LNP identity test. More importantly, the disclosed methods include the ability to characterize the stability of LNP's which can be used to support process and formulation development, for example for LNP-based mRNA vaccines.

### BRIEF DESCRIPTION OF THE DRAWINGS

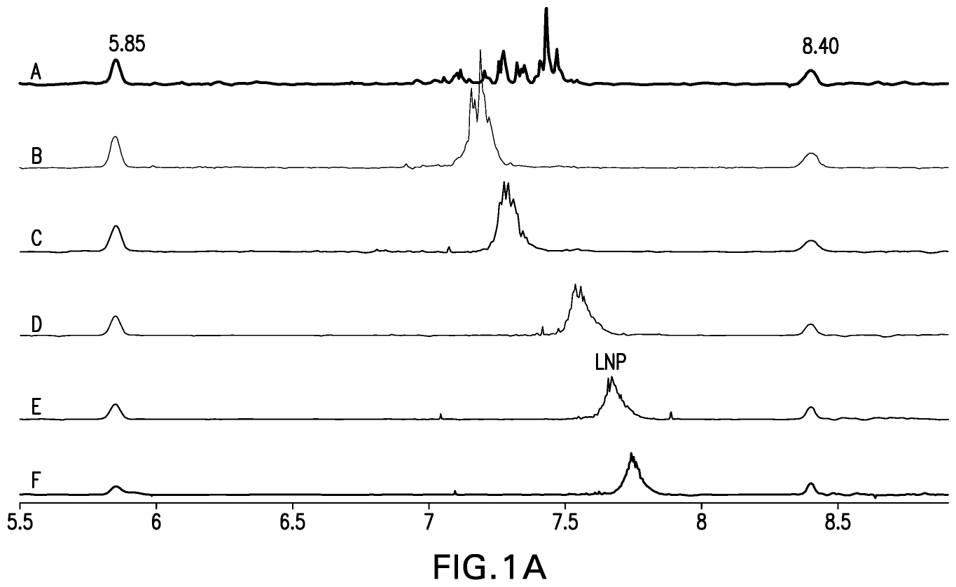

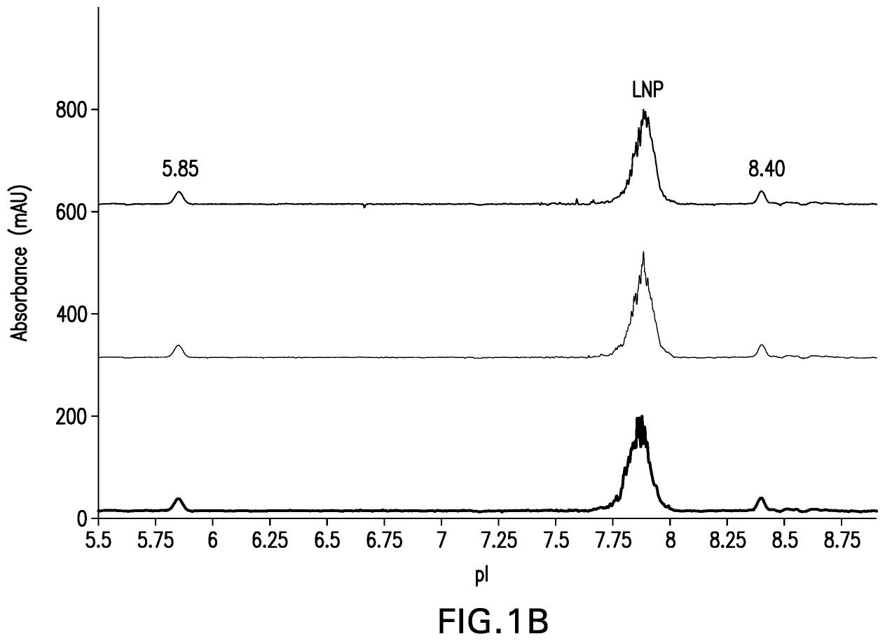

FIG. 1A: Electropherograms of an exemplary LNP using various ampholytes and additives.
Traces A and B show a high background or precipitation and LNP containing sharp peaks using the broad pH range Servalyt™ 2-9 and 3-10 Pharmalyte® ampholytes, respectively. Trace C shows a focused LNP with an apparent pI of ˜7.3 using 3-10 Pharmalyte ampholytes containing 10% glycerol. Trace D uses a 1:2 mixture of narrow pH range and broad pH range (5-8; 3-10) with 10% glycerol. Trace E uses a 2:1 mixture of narrow pH range and broad pH range (5-8; 3-10) with 10% glycerol. Trace F uses 5-8 Pharmalyte® ampholytes containing 10% glycerol. The pI of the LNP shifts to ˜7.6-7.8 in traces D, E, and F. Two pI markers are 5.85 and 8.40.

FIG. 1B: Electropherogram of an LNP prepared in triplicate.
An LNP sample was prepared in triplicate for the icIEF experiment. The LNP has an apparent pI of ˜7.89 and peak shape was consistent for the three replicates.

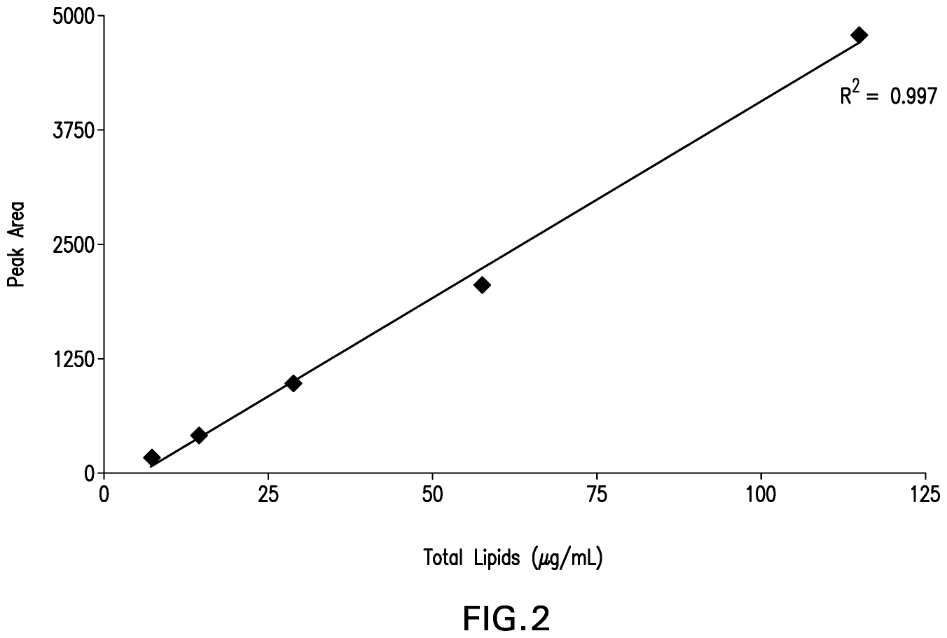

FIG. 2: Calibration curve of LNP ranging from 7-115 μg/mL of total lipids.
LNP samples were diluted in cIEF amphoyte mixtures from 0.56 to 9.0 μg/mL of mRNA (equivalent to 7.2 to 115 μg/mL of total lipid). The area under the peak for each sample was determined and ploted. This linear range has a coefficient of determination (R²)≥0.997 demonstrating the linearity and the ability of this technology to perform quantitative analysis.

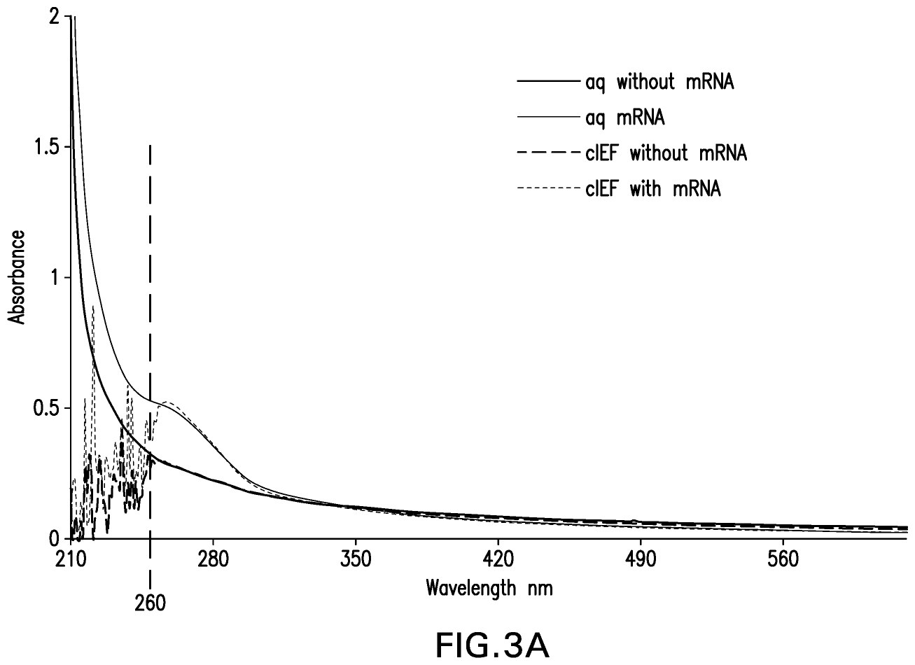

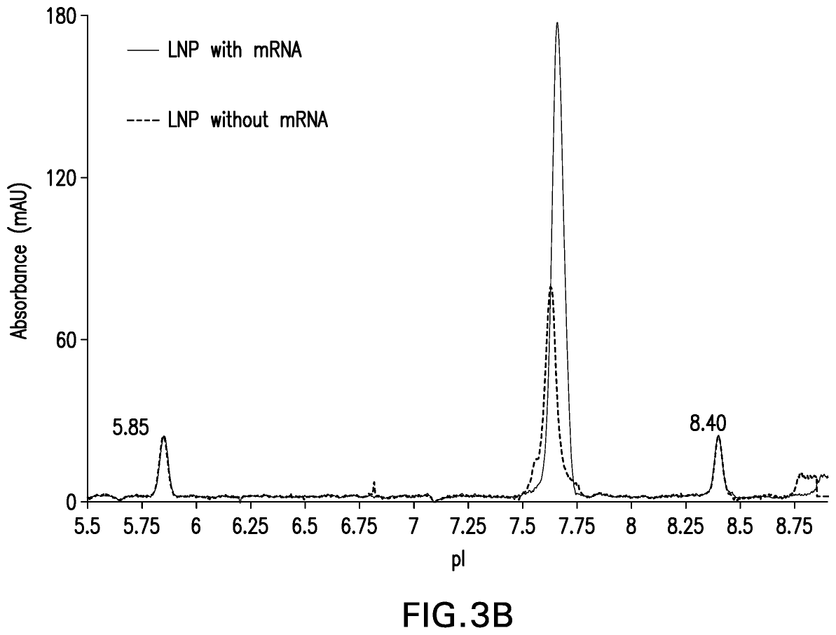

FIG. 3A: UV Absorbance of LNPs in both aqueous and cIEF ampholyte mixtures.
LNPs formulated with mRNA were tested in aqueous Tris buffer ( [symbol] trace) and cIEF ampholyte mixture ( [symbol] trace). LNP formulated without mRNA were tested in aqueous Tris buffer ( [symbol] trace) and cIEF ampholyte mixture ( [symbol] trace). LNPs containing mRNA show an absorbance max at 260 nm compared to LNP without mRNA, which lack a peak at 260 nm. Both aqueous and cIEF ampholyte mixtures absorbance traces were identical when comparing the wavelengths above 260 nm demonstrating that both LNP with or without mRNA LNPs are stable and intact in the final cIEF ampholyte mixture.

FIG. 3B: Electropherogram of LNP formulated with and without mRNA.
LNPs without mRNA (dashed trace) have a similar pI to LNPs formulated with mRNA (solid trace). The pI of both LNPs is ˜7.6-7.7. Two pI markers were 5.85 and 8.40.

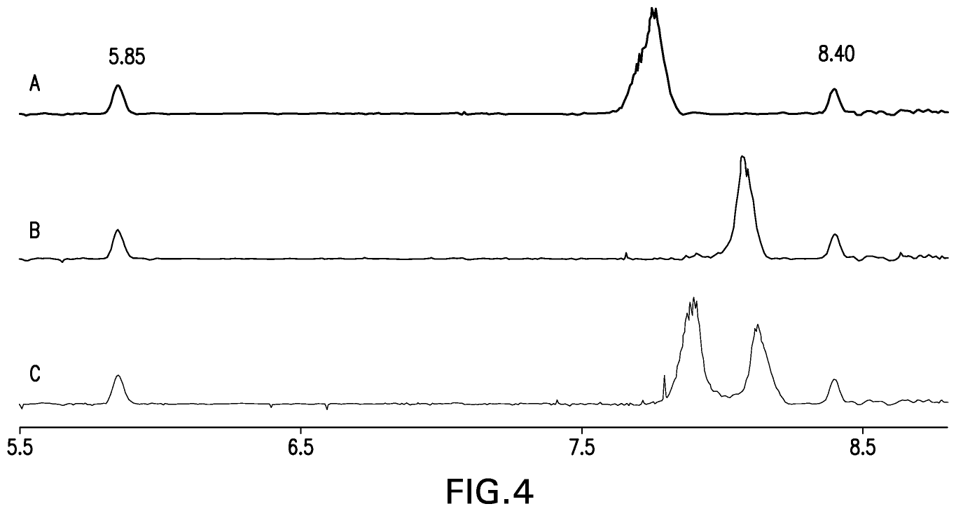

FIG. 4: LNP with different cationic lipids have different pI values.
Trace A shows an LNP containing the cationic lipid Compound B, this LNP has a pI value of 7.6-7.7. Trace B shows an LNP containing the cationic lipid Compound A which has a pI of 8.1. Trace C shows with cIEF separation of a mixture of LNP containing different cationic lipids, namely Compound A and Compound B is possible. Two pI markers are 5.85 and 8.40.

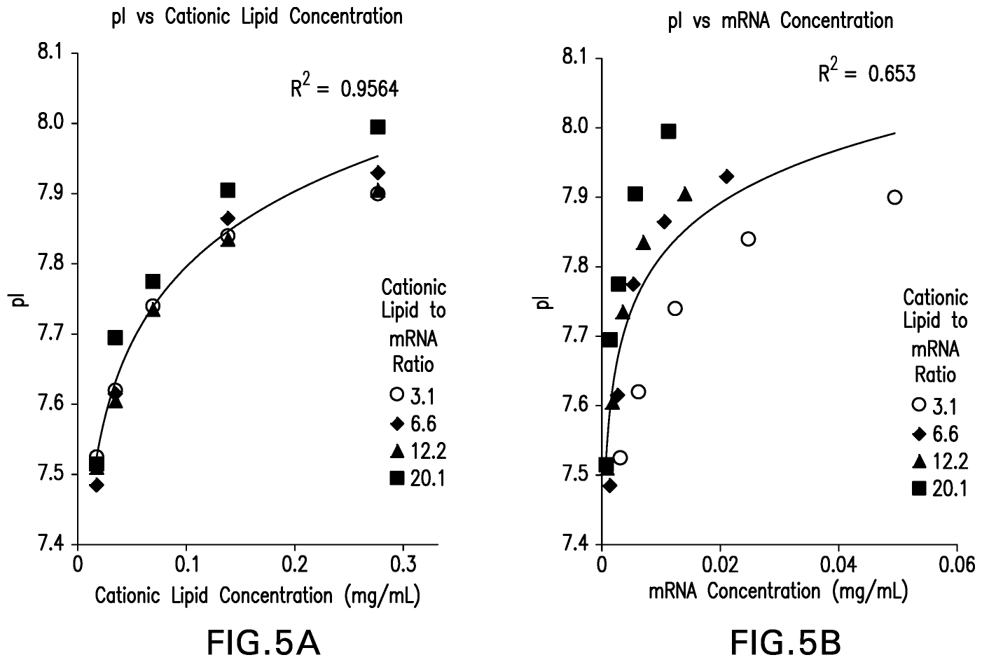

FIGS. 5A & 5B: LNP pI is a function of cationic lipid concentration:
Different LNP batches were formulated to contain different cationic lipid to mRNA ratios. These LNPs were then diluted to five different lipid concentrations and subjected to icIEF. The pI (y-axis) was plotted against cationic lipid concentration (5A) and the mRNA concentration (5B). A strong correlation of pI to cationic lipid concentration was observed (r²=0.956; logarithmic fit) (5A) compared to a weaker correlation of pI to mRNA concentration (r²=0.653; logarithmic fit) (5B).

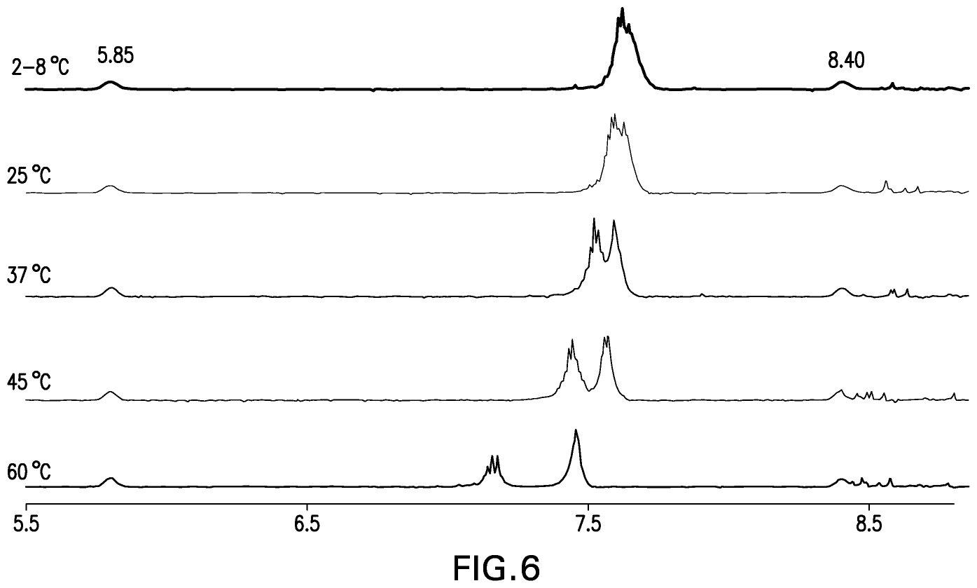

FIG. 6: Stability of LNP with mRNA monitored by icIEF.
The LNPs containing mRNA were exposed to elevated temperatures for 24 hours. Electropherogram of the LNP control that was stored at 2-8° C. shows uniform peak shape with a pI of ˜7.7. The LNP peaks became more acidic and split into 2 distinct peaks as the temperature increased to 25, 37, 45, and 60° C. Two pI markers are 5.85 and 8.40.

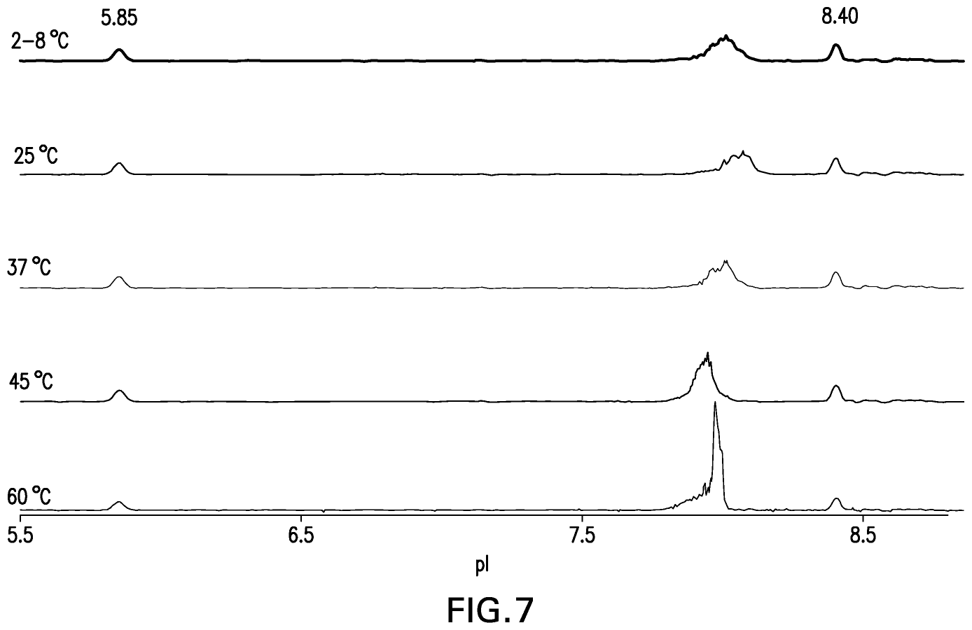

FIG. 7: Stability of LNP without mRNA monitored by icIEF.
The LNP stability experiment was repeated using LNPs that were formulated without mRNA. LNPs without mRNA showed a different degredation pattern compared to the mRNA containing LNPs. At 45° C. after one day, the entire empty LNP peak showed an acidic shift. A sample stressed at 60° C. for one day showed an uncharacteristic peak profile containing a sharp spike in absorbance, which may indicate LNP destabilization. Unlike the mRNA containing LNPs, these preparations did not show splitting into two peaks or generation of acidic variants, indicating that the previously seen acidic peak may contain the negatively charged mRNA.

### DETAILED DESCRIPTION OF THE INVENTION

Advances in drug and vaccine delivery using lipid nanoparticles (LNP) have gained momentum over the past decade as an alternative drug delivery system. Several LNP delivery systems have been approved clinically to deliver small molecule drugs and currently LNP's are being evaluated for delivery of nucleic acids such as siRNA, messenger RNA (mRNA), and plasmid DNA (pDNA) for both therapeutic and vaccine purposes. LNPs for encapsulation of nucleic acids typically comprise three or four lipid components: (1) an ionizable amino lipid (cationic lipid), (2) a zwitterionic phospholipid, such as 1,2-distearoyl-sn-glycero-3-phosphocholine (DSPC) or 1,2-dipalmitoyl-sn-glycero-3-phosphocholine (DPPC), (3) a neutral lipid such as cholesterol, and (4) a polyethylene glycol-lipid (PEG-lipid). The ionizable cationic lipid can play a role, for example, in nucleic acid delivery to cells, by mediating cytosolic delivery of the nucleic acid through facilitated endosomal escape after LNP endocytosis. Neutral lipids, such as DSPC, DPPC, and cholesterol, can modulate the fluidity and phase behavior of the LNP. PEG-lipids can improve particle circulation half-life and systemic exposure.

LNPs are produced through a self-assembly process and can be made to have a particle size range from 70-110 nm depending on the target delivery purpose. The LNP can have a complex structure with respect to particle size, polydispersity, lipid composition, particles morphology, surface hydrophobicity/hydrophilicity, and surface charge. These attributes can affect the uptake of LNP and release of the payload (e.g., nucleic acid) in various cell types. The surface charge may also be correlated with cell toxicity. The FDA also recommends the physiochemical characterization of liposomes, including a stability assessment. LNP size and morphology can be characterized using techniques such as dynamic light scattering (DLS), cryo-electron microscopy (Cryo EM), high performance size exclusion chromatography (HP-SEC), and asymmetric flow field-flow fractionation. However, there is a lack of tools available to measure the surface charge of LNPs. Currently zeta potential is the only available method to measure surface charge of LNP.

Capillary electrophoresis (CE) has been applied to study different large particles such as bacteria, viruses, colloidal/nanoparticles, and polymeric particles. Earlier publications have described traditional isoelectric focusing methods to analyze colloidal nanoparticles and gold nanoparticles. However, these gel-based electrophoretic techniques are labor intensive and qualitative in nature. Liposome protein interactions have been studied using imaged capillary isoelectric focusing (icIEF). However, no prior methods have been found that have explored imaged capillary isoelectric focusing for characterizing LNPs.

The inventors have now discovered methods of charactering LNPs using imaged capillary isoelectric focusing. The methods disclosed herein may be used with empty LNPs or LNPs encapsulating a payload. The payload can comprise, without limitation, a small molecule, a peptide, or nucleic acid payload. The nucleic acid payload can comprise DNA. The nucleic acid payload can comprise RNA (e.g., mRNA, siRNA, dsRNA, pRNA). These methods enable the measurement of the isoelectric poing (pI) of LNPs, and are capable of distinguishing the pI of LNPs manufactured with different ionizable lipids (e.g., different cationic lipids). In addition, these methods are quantitative and stability-indicating. Accordingly, these methods are useful for process and formulation development and quality control for drug and vaccine products incorporating LNP technology.

Various embodiments are disclosed herein. The description herein is exemplary of the invention and is not intended to limit the scope, applicability, or configuration in any way. Various changes to the described embodiments may be made in the function and arrangement of the elements described herein without departing from the scope of the invention.

As used herein, the singular forms “a”, “an”, and “the” include the plural forms unless the context clearly indicates otherwise. Additionally, the term “includes” means “comprises.”

Unless otherwise indicated, the numberical parameters set forth are approximations that may depend on the desired properties sought and/or limits of detection under standard test conditions/methods.

As used herein, the term “anionic lipid” refers to a lipid species that carries a net negative charge at a selected pH, such as physiological pH. These lipids include, but are not limited to, phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglyeerol (POPG), and other anionic modifying groups joined to neutral lipids.

As used herein, the term “cationic lipid” refers to a lipid species that carries a net positive charge at a selected pH, such as physiological pH. Those of skill in the art will appreciate that a cationic lipid can be an ionizable lipid, such as an ionizable cationic lipid. Such lipids include, but are not limited to, N,N-dioleyl-N,N-dimethylammonium chloride (“DODAC”); N-[1-(2,3-dioleyloxy)propyl]-N,N,N-trimethylammonium chloride (“DOTMA”); N,N-distearyl-N,N-dimethylammonium bromide (“DDAB”); N-(2,3dioleoyloxy)propyl)-N,N,N-trimethylammntonium chloride (“DODAP”); 3-(N-(N,N-dimethylaminoethane)-carbam-oyl)cholesterol (“DC-Chol”) and N-(1,2-dimyristyloxyprop-3-yl)-N,N-dimethyl-N-hydroxy ethyl ammonium bromide (“DMRIE”). Additionally, a number of commercial preparations of cationic lipids are available which can be used in the present invention. These include, for example, LIPOFECTIN® (commercially available cationic lipid nanoparticles comprising DOTMA and 1,2-dioleoyl-sn-3-phosphoethanolamine (“DOPE”), from Gibco/BRL, Grand Island, N.Y., USA); LIPOFECTAMINE® (commercially available cationic lipid nanoparticles comprising N-(1-(2,3dioleyloxy)propyl)N-(2-(sperminecarboxamido)ethyl)-N,N-dimethyla-ammonium trifluoroacetate (“DOSPA”) and (“DOPE”), from (Gibco/BRL); and TRANSFECTAM® (commercially available cationic lipids comprising diocmdecylamidoglycyl carboxyspermine (“DOGS”) in ethanol from Promega Corp., Madison, Wis., USA). The following lipids are cationic and have a positive charge at below physiological pH: DODAP, DODMA, DMDMA, 1,2-DiLinoleyloxy-N,N-dimethylaminopropane (DLinDMA), 4-(2,2-diocta-9,12-dienyl[8 1,3]dioxolan-4-ylmethyl)-dimethylamine, DLinKDMA (WO 2009/132131 A1), DLin-K-C2-DMA (WO2010/042877), DLin-M-C3-DMA (WO2010/146740 and/or WO2010/105209), 2-{4-[(3β)-cholest-5-en-3-yloxy]butoxy}-N,N-dimethyl-3-[(9Z,12Z)-oct-adeca-9,12-dienlyloxyl]propan-1-amine) (CLinDMA), and the like.

As used herein, “consists essentially of” means that the composition may include small amounts (i.e., less than 5 wt %) of other components that do not materially alter the composition, such as other excipients.

As used herein, the term “encapsulation” refers to the process or result of confining one or more payloads or agents, such as one or more nucleic acids, within a nanoparticle. As used herein, the terms “encapsulation” and “loading” can be used interchangeably.

As used herein, an “ionizable lipid” is a lipid that has a first charge at a first pH and a second charge at a second pH. Ionizable lipids include lipids with modulated pKa values, such that the ionizable lipid is cationic at a pH below the pKa of the lipid but is neutral or near-neutral in charge at a pH above the pKa of the lipid.

As used herein, the term “isoelectric point,” is known to those skilled in the art, and means the pH at which a molecule has no net electrical charge. In the context of LNP's, the lipid components (e.g., ionizable lipids), and, if present, the payload (e.g., RNA or DNA constructs) may have defined isolelecric points in their isolated state. The isoelectric point of each component may be altered by its surrounding environment, including as formulated with the other components in the LNP.

As used herein, the term “isolectric focusing (IEF)” is known to those skilled in the art, and means a technique for separation of components (e.g., molecules, proteins, or compositions) in an electric field according to their isoelectric point (pI). Traditional, slab-gel IEF techniques involve adding an ampholyte solution into immobilized pH gradient (IPG) gels. Capillary isoelectric focusing (cIEF) eliminates the need for an IPG gel, and is normally performed using an instrument with a capillary and a single-point, on-column detector. First, a solution of carrier ampholytes and components to be analyzed is introduced into the capillary column and a voltage is applied to create a pH gradient by an isoelectric stacking of the carrier ampholytes. Then the components to be analyzed focus into narrow zones at different positions inside the capillary corresponding to their different PI. Finally, the focused zones are mobilized (e.g., by electrophoretic force, hydrodynamic flow, or electroosmotic flow) to pass through the detection point. Imaged capillary isoelectric focusing (icIEF) improves on cIEF by eliminating the mobilization step and instead imaging the capillary in real time (e.g., with a CCD camera). As used herein, the term “lipid” refers to any of a group of organic compounds that are esters of fatty acids and are characterized by being insoluble in water or having low solubility in water but may be soluble in many organic solvents. They can be divided in at least three classes: (1) “simple lipids,” which include, e.g., fats and oils as well as waxes; (2) “compound lipids,” which include, e.g., phospholipids and glycolipids; and (3) “derived lipids,” which include, e.g., steroids.

As used herein, the term “lipid nanoparticle” or “LNP” refers to a lipid composition that forms a particle having a length or width measurement (e.g., a maximum length or width measurement) between 10 and 1000 nanometers. In some embodiments, a lipid nanoparticle is capable of being used to deliver a payload (e.g., a therapeutic agent, such as a nucleic acid). In some embodiments, the lipid composition includes a lipid-defined interior volume in which a payload is encapsulated. In some embodiments, a lipid nanoparticle includes an interior volume that is encapsulated by amphipathic lipid bilayers (e.g., single; unilamellar or multiple; multilamellar). In some embodiments, a lipid nanoparticle forms a lipid aggregate in which the encapsulated payload is contained within a relatively disordered lipid mixture. In some embodiments, a lipid nanoparticle forms a lipid aggregate in which the payload is contained within a relatively ordered lipid mixture, forming non-lamellar structures (e.g. micelle, hexagonal, etc.). In some embodiments, the lipid nanoparticle is empty. Such empty lipid nanoparticles can have any of the afore mentioned structures.

As used herein “pKa” is defined as the negative logarithm of the ionization constant (K) of an acid, which is the pH of a solution in which half of the acid molecules are ionized.

As used herein, the term “stability” is used to describe the LNP, and means the ability or tendency of the components of the LNP to retain their structural integrity and resist degradation and or aggregation.

Disclosed herein is a robust method to measure the isoelectric points of LNPs containing ionizable lipids (e.g., anionic or cationic lipids) using imaged capillary isoelectric focusing. The methods disclosed herein can involve the use of a stabilizer (e.g., glycerol or sucrose) during icIEF. The inventors have found that the stabilizer can help maintain, but is not required for, LNP stability in the separation matrix. The LNPs can contain one species of ionizable lipid (e.g., one species of cationic lipid) or various different ionizable lipids (e.g., two or more species of cationic lipid). In an embodiment, the method may be used for quantitation of LNP. In another embodiment, the method may be used for analyzing different LNPs containing different ionizable (e.g., cationic) lipids as a potential test for LNP identity. Still in another embodiment, the method is a stability indicating assay which can be used to support process and formulation development for drug or vaccine products incorporating LNP technology (e.g., LNP-based mRNA, dsRNA, siRNA, and/or peptide therapeutics or vaccines). In another embodiment, image capillary isoelectric focusing is used to provide a quick screening of many different ionizable amino lipid-based LNP since it is known that surface charge may be correlated with toxicity.

Accordingly, disclosed herein are methods of separating lipid nanoparticles (LNPs) according to their isoelectic points, the methods comprising applying a separating voltage to a separation matrix comprising carrier ampholytes and the LNPs for a sufficient time to separate the LNPs according to their isoelectic points. The separation matrix may further comprise a stabilizer (e.g., glycerol or sucrose), methycellulose, pI markers, or any combination thereof. The methods can be performed in a capillary. The separation matrix can be imaged (e.g., by detecting

UV absorbance at 280 nm) following separation to produce an electropherogram. The electropherogram can be compared to reference electropherograms, for example, to evaluate batch consistency, changes in LNP stability, or to identify or verify the identity of ionizable lipids (e.g., anionic or cationic lipids) in an LNP. The measured peak area in the electropherogram can be used to calculate lipid levels. Specific embodiments of the invention are detailed below, which are followed by the experimental section.

### Specific Embodiments

Embodiment 1. A method of separating lipid nanoparticles (LNPs) according to their isoelectic points, the method comprising applying a separating voltage to a separation matrix comprising carrier ampholytes and the LNPs for a sufficient time to separate the LNPs according to their isoelectic points.

Embodiment 2. The method of embodiment 1, wherein the separation matrix further comprises a stabilizer.

Embodiment 3. The method of embodiment 2, wherein the stabilizer is glycerol or sucrose.

Embodiment 4. The method of embodiment 2, wherein the stabilizer is glycerol.

Embodiment 5. The method of embodiment 2, wherein the stabilizer is sucrose.

Embodiment 6. The method of any one of embodiments 2-5, wherein the stabilizer is present in the separation matrix at a level of from: 1-40%, 1-30%, 1-20%, 5-15%, 6-14%, 7-13%, 8-12%, 9-11%, or 9.5-10% (w/v or v/v).

Embodiment 7. The method of any one of embodiments 2-5, wherein the stabilizer is present in the separation matrix at 5-15% (w/v or v/v).

Embodiment 8. The method of any one of embodiments 2-5, wherein the stabilizer is present in the separation matrix at 9-11% (w/v or v/v).

Embodiment 9. The method of any one of embodiments 2-5, wherein the stabilizer is present in the separation matrix at a level within 10% of: 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% (w/v or v/v).

Embodiment 10. The method of any one of embodiments 2-5, wherein the stabilizer is present in the separation matrix at about 10% (w/v or v/v).

Embodiment 11. The method of any one of embodiments 1-10, wherein the separation matrix further comprises methylcellulose.

Embodiment 12. The method of embodiment 11, wherein the methylcellulose is present in the separation matrix at 0.01%-5% w/v, 0.05%-1% w/v, 0.1%-0.5% w/v, 0.15%-0.35% w/v, 0.18%-0.3% w/v, or 0.2%-0.25% w/v.

Embodiment 13. The method of embodiment 11, wherein the methylcellulose is present in the separation matrix at 0.2%-0.25% w/v.

Embodiment 14. The method of embodiment 11, wherein the methylcellulose is present in the separation matrix at an amount within 10% of: 0.01% w/v, 0.02% w/v, 0.03% w/v, 0.04% w/v, 0.05% w/v, 0.06% w/v, 0.07% w/v, 0.08% w/v, 0.09% w/v, 0.1% w/v, 0.11% w/v, 0.12% w/v, 0.13% w/v, 0.14% w/v, 0.15% w/v, 0.16% w/v, 0.17% w/v, 0.18% w/v, 0.19% w/v, 0.2% w/v, 0.21% w/v, 0.22% w/v, 0.23% w/v, 0.24% w/v, 0.25% w/v, 0.26% w/v, 0.27% w/v, 0.28% w/v, 0.29% w/v, 0.3% w/v, 0.31% w/v, 0.32% w/v, 0.33% w/v, 0.34% w/v, 0.35% w/v, 0.36% w/v, 0.37% w/v, 0.38% w/v, 0.39% w/v, 0.4% w/v, 0.41% w/v, 0.42% w/v, 0.43% w/v, 0.44% w/v, 0.45% w/v, 0.46% w/v, 0.47% w/v, 0.48% w/v, 0.49% w/v, or 0.5% w/v. Embodiment 15. The method of embodiment 11, wherein the methylcellulose is present in the separation matrix at about 0.22% w/v.

Embodiment 16. The method of any one of embodiments 1-15, wherein the separation matrix further comprises one or more pI markers.

Embodiment 17. The method of any one of embodiments 1-15, wherein the separation matrix further comprises two or more pI markers.

Embodiment 18. The method of any one of embodiments 1-17, wherein the carrier ampholytes form a pH gradient when the voltage is applied.

Embodiment 19. The method of embodiment 18, wherein the pH gradient has a pH range from: 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10.

Embodiment 20. The method of embodiment 18 or 19, wherein the pH gradient is linear.

Embodiment 21. The method of embodiment 18 or 19, wherein the pH gradient is sigmoidal.

Embodiment 22. The method of embodiment 21, wherein the sigmoidal pH gradient is capable of higher resolution separation in a pH sub-range that includes the isoelectric points of the LNPs.

Embodiment 23. The method of any one of embodiments 1-22, wherein the separation matrix is in a capillary.

Embodiment 24. The method of embodiment 23, wherein the capillary is silica.

Embodiment 25. The method of embodiment 23 or 24, wherein the capillary is coated.

Embodiment 26. The method of any one of embodiments 23-25, wherein the capillary is coated with fluorocarbon.

Embodiment 27. The method of any one of embodiments 23-26, wherein the capillary has an internal diameter (ID) within 10% of: 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, or 300 μm.

Embodiment 28. The method of any one of embodiments 23-26, wherein the capillary has an internal diameter (ID) within 10% of 100 μm.

Embodiment 29. The method of any one of embodiments 23-28, wherein the capillary has an outer diameter (OD) within 10% of: 100 μm, 110 μm, 120 μm, 130 μm, 140 μm, 150 μm, 160 μm, 170 μm, 180 μm, 190 μm, 200 μm, 210 μm, 220 μm, 230 μm, 240 μm, 250 μm, 260 μm, 270 μm, 280 μm, 290 μm, 300 μm, 310 μm, 320 μm, 330 μm, 340 μm, 350 μm, 360 μm, 370 μm, 380 μm, 390 μm, or 400 μm.

Embodiment 30. The method of any one of embodiments 23-28, wherein the capillary has an outer diameter (OD) within 10% of 200 μm.

Embodiment 31. The method of any one of embodiments 23-30, wherein the capillary has a length that is within 10% of: 10 mm, 11 mm, 12 mm, 13 mm, 14 mm, 15 mm, 16 mm, 17 mm, 18 mm, 19 mm, 20 mm, 22.5 mm, 25 mm, 27.5 mm, 30 mm, 32.5 mm, 35 mm, 37.5 mm, 40 mm, 42.5 mm, 45 mm, 47.5 mm, 50 mm, 55 mm, 60 mm, 65 mm, 70 mm, 75 mm, 80 mm, 85 mm, 90 mm, 95 mm, 100 mm, 110 mm, 120 mm, 130 mm, 140 mm, 150 mm, 160 mm, 170 mm, 180 mm, 190 mm, 200 mm, 210 mm, 220 mm, 230 mm, 240 mm, 250 mm, 275 mm, 300 mm, 325 mm, 350 mm, 375 mm, 400 mm, 425 mm, 450 mm, 475 mm, or 500 mm.

Embodiment 32. The method of any one of embodiments 23-30, wherein the capillary has a length that is within 10% of: 100 mm.

Embodiment 33. The method of any one of embodiments 1-32, wherein the separating voltage is from: 1 to 1200 V/cm, 100 to 1100 V/cm, 200 to 1000 V/cm, 300 to 900 V/cm, 400 to 800 V/cm, 500 to 700 V/cm, 550 to 650 V/cm, 575 to 625 V/cm, or 590 to 610 V/cm.

Embodiment 34. The method of any one of embodiments 1-32, wherein the separating voltage is from 500 to 700 V/cm.

Embodiment 35. The method of any one of embodiments 1-32, wherein the separating voltage is from 550 to 650 V/cm.

Embodiment 36. The method of any one of embodiments 1-32, wherein the voltage is from 575 to 625 V/cm.

Embodiment 37. The method of any one of embodiments 1-32, wherein the separating voltage is within 10% of: 1 V/cm, 10 V/cm, 25 V/cm, 50 V/cm, 75 V/cm, 100 V/cm, 125 V/cm, 150 V/cm, 175 V/cm, 200 V/cm, 225 V/cm, 250 V/cm, 275 V/cm, 300 V/cm, 325 V/cm, 350 V/cm, 375 V/cm, 400 V/cm, 425 V/cm, 450 V/cm, 475 V/cm, 500 V/cm, 525 V/cm, 550 V/cm, 575 V/cm, 600 V/cm, 625 V/cm, 650 V/cm, 675 V/cm, 700 V/cm, 725 V/cm, 750 V/cm, 775 V/cm, 800 V/cm, 825 V/cm, 850 V/cm, 875 V/cm, 900 V/cm, 925 V/cm, 950 V/cm, 975 V/cm, 1000 V/cm, 1025 V/cm, 1050 V/cm, 1075 V/cm, 1100 V/cm, 1125 V/cm, 1150 V/cm, 1175 V/cm, or 1200 V/cm.

Embodiment 38. The method of any one of embodiments 1-32, wherein the separating voltage is within 10% of 600 V/cm.

Embodiment 39. The method of any one of embodiments 1-38, wherein the sufficient time is from 1-60 min, 2-45 min, 3-30 min, 4-20 min, 5-15 min, 6-10 min, or 7-9 min.

Embodiment 40. The method of any one of embodiments 1-38, wherein the sufficient time is 7-9 min.

Embodiment 41. The method of any one of embodiments 1-40, further comprising pre-focusing the separation matrix at a reduced voltage prior to applying the separating voltage.

Embodiment 42. The method of embodiment 41, wherein the reduced voltage is 1-10%, 5-15%, 10-20%, 15-25%, 20-30%, 25-35%, 30-40%, 35-45%, 40-50%, 45-55%, 50-60%, 55-65%, 60-70%, 65-75%, 70-80%, 75-85%, 80-90%, 85-95%, or 90-100% of the separating voltage.

Embodiment 43. The method of embodiment 41, wherein the reduced voltage is 45-55% of the separating voltage.

Embodiment 44. The method of any one of embodiments 41-43, wherein the reduced voltage is applied for: 1-10 sec., 5-15 sec., 10-20 sec., 15-25 sec., 20-30sec., 25-35 sec., 30-40 sec., 35-45 sec., 40-50 sec., 45-55 sec., 50-60 sec., 55-65 sec., 60-70 sec., 65-75 sec., 70-80 sec., 75-85 sec., 80-90 sec., 85-95 sec., 90-100 sec., 95-105 sec., 100-110 sec., 105-115 sec., or 110-120 sec.

Embodiment 45. The method of any one of embodiments 41-43, wherein the reduced voltage is applied for 55-65 sec.

Embodiment 46. The method of any one of embodiments 1-45, wherein the LNPs comprise, individually, one or more cationic lipid species, one or more non-cationic lipid species, cholesterol, one or more PEG-lipids, or a combination thereof.

Embodiment 47. The method of any one of embodiments 1-46, wherein the LNPs comprise, individually, one or more encapsulated nucleic acids, peptides, or small molecules.

Embodiment 48. The method of any one of embodiments 1-46, wherein the LNPs comprise, individually, one or more encapsulated nucleic acids.

Embodiment 49. The method of any one of embodiments 1-46, wherein the LNPs comprise, individually, one or more encapsulated mRNA species.

Embodiment 50. The method of any one of embodiments 1-46, wherein the LNPs comprise, individually, one or more encapsulated siRNA species.

Embodiment 51. The method of any one of embodiments 1-50, further comprising imaging the separation matrix after the sufficient time to produce an electropherogram.

Embodiment 52. The method of embodiment 51, wherein imaging the separation matrix comprises detecting UV absorbance at 280 nm.

Embodiment 53. The method of embodiment 51 or 52, further comprising measuring a peak area corresponding to LNPs having a selected pI in the electropherogram and comparing the peak area to a calibration curve to calculate a total lipids concentration.

Embodiment 54. The method of embodiment 51 or 52, further comprising comparing the electropherogram to a reference electropherogram produced under identical conditions.

Embodiment 55. The method of embodiment 54, wherein the reference electropherogram was produced from the same batch of LNPs as the LNPs in the electropherogram, and wherein an acidic shift or altered peaks in the electropherogram indicate a change in LNP stability.

Embodiment 56. The method of embodiment 54, wherein the reference electropherogram was produced from a reference batch of LNPs having the same lipid composition as the LNPs in the electropherogram, and wherein a significant difference between the electropherograms indicates a manufacturing problem for the LNPs.

Embodiment 57. The method of any one of embodiments 53-56, wherein the LNPs in the separation matrix comprise a normalized level of ionizable lipids.

Embodiment 58. The method of any one of embodiments 53-56, wherein the LNPs in the separation matrix comprise a normalized level of cationic lipids.

Embodiment 59. A method for determining the isoelectric point (pI) of lipid nanoparticles (LNP) having a cationic lipid, comprising the steps of (a) subjecting a composition comprising LNP to isoelectric focusing, and (b) measuring total charge of the LNP to determine the isoelectric point, wherein observed isoelectric point is proportional to LNP diameter and total concentration.

Embodiment 60. The method according to embodiment 59, wherein the LNP diameter ranges from about 60 to about 150 nm, or about 70 to about 140 nm.

Embodiment 61. The method according to embodiment 59, wherein the LNP diameter is about 70 to about 140 nm.

Embodiment 62. The method according to embodiment 59, wherein the total LNP concentration of the compositions ranges from 7 to 115 μg/mL.

Embodiment 63. The method according to embodiment 59, wherein LNP diameter is about 70 to about 140 nm and LNP concentration is about 7 to 115 μg/mL.

Embodiment 64. The method according to embodiment 59, wherein the LNPs are formulated with RNA, DNA, or peptides.

Embodiment 65. The method according to embodiment 64, wherein the RNA is selected from the group consisting of mRNA, dsRNA, and siRNA.

Embodiment 66. The method according to embodiment 59, wherein the lipid nanoparticles are formulated with RNA selected from the group consisting of mRNA, dsRNA, and siRNA.

Embodiment 67. The method according to embodiment 66, wherein LNP diameter is about 70 to about 140 nm, and the total concentration of the composition ranges from 7 to 115 pg/mL.

Embodiment 68. The method according to embodiment 59, for use in the manufacture of LNP-based mRNA vaccines or therapeutics.

Embodiment 69. A method for quantifying total lipid amount (w/v %) of lipid nanoparticle (LNP) comprising the steps of, (a) subjecting a composition comprising test LNPs to isoelectric focusing, (b) measuring total charge of the test LNPs, (c) determining the isoelectric point (pI) of the test LNPs, (d) measuring peak area of the test LNPs, and (e) comparing peak area of LNP of known lipid amount to the test LNP.

Embodiment 70. A method for separating a composition comprising lipid nanoparticles (LNPs), each of said LNPs comprising one or more same or different cationic lipids, comprising the steps of : (a) subjecting the composition to isoelectric focusing, (b) measuring total charge of different LNPs based upon pKa's of cationic lipids, (c) determining the isoelectric points (pI) of the differently charged LNPs and (d) separating LNPs based upon the pis of the LNPs.

Embodiment 71. A method for determining stability of lipid nanoparticle (LNP) compositions comprising, (a) measuring isoelectric point (pI) of LNP compositions to obtain a baseline pI at a first temperature, (b) exposing the LNP compositions to a second temperature, (c) subjecting said exposed LNP compositions to image isoelectric focusing to obtain a first pI measurement and (d) determining stability based upon the difference in the first pI from the baseline pI.

Embodiment 72. The method according to embodiment 71, wherein the LNP diameter is about 70 to about 140 nm and LNP concentration of the composition is about 7 to 115 μg/mL.

### EXAMPLES

The invention now being generally described will be more readily understood by reference to the following examples which are included merely for purposes of illustration of certain aspects and embodiments, and are not intended to limit the invention in any way. General: All methylcellulose (MC) containing solutions, icIEF fluorescence calibration instrument, system suitability standards, the pI markers (5.85 and 8.40), Seryalyt™ 2-9 ampholytes, and the icIEF cartridge were obtained from ProteinSimple (Santa Clara, USA). Pharmalyte® ampholytes pH 3 -10 and pH 5-8 were purchased from GE Healthcare (Uppsala, Sweden). Glycerol and sucrose were purchased from Sigma-Aldrich (St. Louis, Mo., USA).

### Preparative Example 1

### Lipid Nanoparticle (LNP) Preparation.

LNPs may be made by methods generally known in the art. See for example, Gindy, M. E., et al., Mol. Pharm. 2014, 11, 4143-4153; Zhang, J., et al., Anal. Chem. 2012, 84, 6088-6096; Gindy, M. E., et al, Expert Opin. Drug Delivery 2012, 9 (2), 171-182; US2013/0039799, U.S. Pat. No. 9,404,127; and U.S. Pat. No. 8,058,069. Briefly, LNPs may be formulated using, for example, a combination of Tee-mixing and filtration processes. The Tee-mixing steps form nucleic acid encapsulated LNPs through nanoprecipitation by combining a lipid solution in an organic solvent (e.g., ethanol) with an aqueous nucleic acid solution. Empty LNPs can be formulated by using a nucleic acid-free aqueous solution. The filtration (e.g., ultrafiltration) step then concentrates the LNPs, removes excess solvent from the formulation, and exchanges the LNPs into final formulation buffer.

With some modification, siRNA encapsulated LNPs can be prepared according to the rapid precipitation process, Gindy, M. E., et al, Expert Opin. Drug Delivery 2012, 9 (2), 171-182. Specifically, siRNA encapsulated LNPs are assembled by micromixing of an organic solution of lipids with an aqueous solution containing siRNA duplexes. The lipids solutions are prepared by dissolving amino lipid, cholesterol, phospholipid and PEG2000-DMG, in a molar ratio of 58-50:30-38:10:2-1, in ethanol. siRNA duplexes are prepared in an aqueous sodium citrate buffer (20 mM, pH 5) at a concentration targeting a N:P ratio of 6. Reagent solutions are preheated to 30-40° C. and delivered at nearly equal volumetric flow rates to the inlet of a confined volume T-mixer device (ID 0.5 mm) using syringe pumps (Harvard Apparatus PHD 2000, Holliston, Mass.). The ethanol and aqueous citrate solutions are delivered to the inlet of a T-mixer with a total flow rate from 100 to 150 mL/min. The mixed LNP solution are diluted into an equal volume of citrate buffer 20 mM citrate, 300 mM NaCl, pH 6, and preheated to 30-40° C. The resulting LNP suspension are further mixed with a phosphate buffered saline (PBS) at pH of 7.5 at a ratio of 1:1 vol:vol. Following dilutions, LNPs are incubated for 0.5-1 h at 30-40° C. The residual ethanol is removed and the external buffer exchanged into PBS via tangential flow diafiltration a hollow fiber PES membrane (Spectrum Laboratories, Rancho Dominguez, Calif.). The resulting LNPs are concentrated to target concentration of 2-30 mg/mL total lipids, sterilized via filtration through 0.45 and 0.2 μm sterile filters (Pall Corp.), and dispensed into sterile vials under aseptic conditions.

The LNP preparations used in the examples below include: i) with, or without (empty LNP) mRNA, drug substance, ii) a cationic lipid referred to as Compound A or Compound B, each of which is an ionizable lipid that complexes with the mRNA to promote the formation of the LNPs, and iii) one or more of additional lipids such as cholesterol; 1,2-Distearoyl-sn-glycero-3-phosphorylcholine (DSPC); and 1,2-Dimyristoyl-sn-glycerol, methoxypolyethyleneglycol (PEG-DMG) that contribute to the overall pharmaceutical properties of the LNP. A lipid stock solution was prepared by dissolving the cationic lipid, cholesterol, phospholipid, and PEG-DMG in ethanol in a molar ratio of 50-58:30-38:10:1-2.

### Preparative Example 2

### Imaged Capillary Isoelectric Focusing

IcIEF Sample Preparation

An ampholyte solution is prepared by combining 2 parts of Pharmalyte® ampholytes pH 5-8 with 1 part of Pharmalyte® ampholytes pH 3-10. The icIEF sample is prepared by combining 70 μLof 0.5% methylcellulose, 8 μL of the combined ampholytes solution, 40 μL of glycerol or sucrose, 1 μL of each pI marker 5.85 and 8.40 with various volumes of LNP to make standard curves and various amount of water to obtain a final volume of 160 μL for each sample. The pI markers are used to establish a pI reference, so that the pI of the test sample LNP can be determined. The samples are centrifuged at 5000×g for 5 min before 120 μL is transferred to the 96-well plate. Lastly the plate is covered with pierceable film, centrifuged at 1000×g for 5 minutes and then placed into the icIEF instrument.

The icIEF separation cartridge is 50 mm in length, 100 μm ID×200 μm OD silica capillary coated with fluorocarbon (Protein Simple, Santa Clara, Calif., USA). The catholyte consists of 0.1 M NaOH in 0.1% methylcellulose and the analyte is 0.08 M phosphoric acid in 0.1% methylcellulose. Additional operational details are described in vendor instruction manual. All other needed reagents—such as system suitability standard, fluorescence calibration standard, 0.5% methylcellulose—were prepared according to vendor recommendation. The capillary is automatically calibrated with a fluorescence standard and pre conditioned with a system suitability control to ensure the capillary is not defective. The samples are pre-focused for 1 min at 1500 V followed by a focusing for 8 min at 3000 V. All electropherograms are collected and detected with UV at 280 nm wavelength. All data analyses are performed using Protein Simple software called Compass for iCE.

### Example 1

IcIEF with 3-10 Ampholyte:

Development of icIEF for LNP was initially started with broad range ampholytes to find the pI of LNP (comprising Compound B) and then narrower pH range ampholytes were evaluated. Briefly, ampholytes mixtures were prepared by combining 8 μL of carrier ampholytes with 70 μL of 0.5% methylcellulose, varying volumes of glycerol or sucrose, 1 μL of each pI marker 5.85 and 8.40 with various volumes of LNP and water to a final volume of 160 μL. First, two different broad range ampholytes (Servalyt® pH 2-9 and Pharmalyte® ampholytes pH 3-10) were tested and compared initially as shown in FIG. 1 electropherogram A and B, respectively. The Servalyt® ampholytes profile show many sharp irreproducible peaks indicating possible LNP precipitation or aggregation. The Pharmalyte® mixture showed an inconsistent broad peak shape. Varying amounts of glycerol (5%, 10%, 20%, and 40%) were added into the ampholytes mixtures. The addition of 10% glycerol helped to consistently and reproducibly focus the LNP as illustrated in FIG. 1 trace C. The higher percentages (20% and 40%) of glycerol noticeably increased the viscosity, thus deceased ability of the LNP to be focused in the tested separation time (data not shown). Lastly 10% sucrose was evaluated as an alternative to glycerol and the LNP peak shape was similar to that of 10% glycerol (data not shown). All subsequent experimental conditions contained 10% glycerol instead of 10% sucrose. Using the broad range ampholytes mixtures the initial observation of LNP pI was approximately 7.3 (FIG. 1A Trace C).

### p Example 2

IcIEF with 3-10/5-8 Ampholyte Mixture: To increase the accuracy of the apparent pI, a narrower range Pharmalyte® amphoylte 5-8 solution was added to the broader range Pharmalyte® ampholytes pH 3-10. As the percentage of the narrow range ampholytes increased from 33% to 66% to 100%, the apparent pI shifted from approximately 7.6 to 7.8 (FIG. 1A Trace D, E, and F, respectively). The peak is relatively broad and not quite homogeneous as normally observed for proteins. This is likely caused by the polydispersity of LNP populations. The observed pI range of 7.6-7.8 is largely driven by pKa of the cationic lipid used for this LNP since the other three lipid components are either neutral (phospholipid) or have no charge (PEG-lipid and cholesterol). Hence it strongly implies that the cationic lipid is indeed at the surface of LNP and there is almost negligible contribution of mRNA.

A 2:1 ratio of broad range (Pharmalyte® ampholytes pH 3-10) to narrow range (Pharmalyte® amphoylte 5-8) ampholytes (as in FIG. 1A, Tace E) was selected for the remaining experiments because of the consistency and reproducibility of the separations (as seen for three individual preparations in FIG. 1B). The focusing time was varied from 6-16 min and indicated 8 min focusing time was adequate for this LNP. Precision of the pI was evaluated with this LNP sample over 16 independent runs; the LNP had an average pI of 7.89±0.028 (RSD<0.4%).

Five concentrations of an LNP sample ranging from 0.56 to 9.0 μg/mL of mRNA (equivalent to 7.2 to 115 μg/mL of total lipids) were separated by icIEF and the pI was determined. Using linear regression analysis the peak area of the five calibration standards was plotted against the total lipids amount (μg/mL) (FIG. 2). Such calibration curves can be used to determine the amount of LNP particles in unknown test samples. This linear range has a coefficient of determination (r²)≥0.997 demonstrating the linearity and the ability of this technology to perform quantitative analysis.

### Example 3

### LNP is Stable in Final Ampholytes Mixture.

The icIEF instrument detects the LNP at 280 nm, and the instrument does not allow this wavelength to be altered. To better understand how the LNP was being detected by the icIEF instrument, the absorbance spectrum of LNPs (comprising Compound B) formulated with or without mRNA was measured using an Agilent 8453 ultraviolet-visible spectrophotometer. The spectrum for each formulation was measured from 210 nm to 600 nm in two different matrices: a Tris buffer (10 mM Tris with 10% sucrose) and cIEF ampholytes matrix (one part 3-10 Pharmalyte® ampholytes was mixed with two parts of 5-8 Pharmalyte® ampholytes in the presence of 10% glycerol). The Tris buffer was measured because ampholytes used in cIEF are known to have interference at wavelengths below 280 nm. In addition, the samples prepared in Tris buffer serve as a control spectrum of intact LNPs. FIG. 3A shows UV spectra for each matrix measured from 210 nm to 600 nm.

The UV spectrum for mRNA containing LNP in both aqueous and ampholyte matrices showed an elevated UV abs from 240 to 290 nm, with absorbance maximum at 260 nm. The absorbance at 260 nm is due to the mRNA in the LNP. In addition, the LNP showed significant light scatter throughout the wavelengths collected.

Comparing the UV spectrum for empty (no mRNA) LNPs in aqueous and ampholyte matrices showed significant light scatter throughout the wavelengths collected. No elevated UV Absorbance was observed at 240-290 nm. These data suggest that observed icIEF signal at 280 nm results from a combination of both the mRNA absorbance and light scattering of the ˜100 nm LNP. Thus, mRNA is not needed to obtain a signal at 280 nm.

Lastly LNPs measured in the ampholyte matrix displayed variable signal from 210 to 256 nm. This was expected due to interfering components in the ampholyte matrix resulting in higher background below 250 nm. Regardless, both aqueous and ampholyte matrices absorbance traces were identical after 260 nm demonstrating that both LNP with or without mRNA are stable and intact in the final ampholyte matrix (FIG. 3A).

The apparent pis of LNPs formulated with or formulated without mRNA were subsequently measured by icIEF. FIG. 3B shows that LNPs with and without mRNA have similar pis at ˜7.7. As expected, the peak areas for LNPs containing mRNA are larger than those for LNPs without mRNA. These data further support the conclusion that the observed signal is a combination of both scattered light and mRNA absorbance.

### Example 4

Measuring LNPs with Different Cationic Lipids having Unique pIs:

The LNPs contain several ionizable groups that can contribute to the apparent pI: the phosphate backbone of the mRNA, the cationic lipid, the zwitterionic phospholipid, and potential degradants from the various lipids (e.g. fatty acids resulting from hydrolysis of DSPC or the PEG-lipid). The contribution of the phosphate backbone within the mRNA is negligible as shown FIG. 3B (and FIG. 5, discussed below in Example 5).

In order to further demonstrate that observed LNP pI is dependent on the cationic lipid used in the LNP, a second LNP was evaluated which contained cationic lipid, Compound A. Compound A has an apprioximate 0.4 higher pKa than the Compound B. FIG. 4 Trace A shows an LNP containing Compound B with a pI of ˜7.75. FIG. 4 Trace B shows an LNP containing Compound A with a higher apparent pI of 8.1. This suggests that the pKa of the cationic lipid is the main contributing factor for the observed pI of a cationic lipid-containing LNP. When LNPs containing Compound B are mixed together with LNPs containing Compound A, both are base line resolved as shown in FIG. 4 Trace C. A slight basic shift for both LNP peaks was observed; at this time, the mechanism for this observation is not known. Regardless, the method is capable of separating LNPs by their pI based of the discrete cationic lipids pKa (FIG. 4). These data also demonstrate that the icIEF method can be used to confirm the identity of the cationic lipid present within the LNP.

### Example 5

### LNP pI is a Function of Cationic Lipid Concentration

Sample pI was found to vary when loading different quantities of LNP based on mRNA concentration into the ampholytes mixture; samples of higher LNP concentration display a higher apparent pl. To investigate whether the pI variation was caused by the cationic lipid or the mRNA concentration, both parameters were examined. Four different LNP batches were formulated and each batch contained a different cationic lipid to mRNA ratio (mole/mole). The four LNP batches had cationic lipid to mRNA ratios of 3.1, 6.6, 12.2, and 20.1. The LNP batches were diluted to five different cationic lipid concentrations and subjected to icIEF. The apparent pis for all prepared LNPs were plotted against cationic lipid and mRNA concentrations.

The apparent pI was found to have a strong correlation to cationic lipid concentration with an R2=0.956, using a logarithmic fit (FIG. 5A). The apparent pI has a weaker correlation with an R2=0.653, using a logarithmic fit to mRNA concentration (FIG. 5B). Results of this experiment indicate that icIEF sample loading may be normalized according to cationic lipid concentration (rather than to mRNA concentration) to maintain consistent pI results between different LNP batches. These data correlate well with the hypothesis that the cationic lipid is at the surface of the LNP and the mRNA is located inside of the LNP. This hypothesis is reasonable because the LNP acts as a protective hydrophobic barrier to protect the mRNA.

### Example 6

### Detecting LNP Stability:

The icIEF method can detect changes in LNP stability upon heat stress as shown in FIGS. 6 and 7. In FIG. 6, when an mRNA containing LNP sample was heat-stressed at 37° C. for 24 hours, the entire LNP profile shifts to lower apparent pI values and a new peak is detected (analogous to acidic variants in the context of protein analsyis). The “acidic variants” became more acidic as the heat stressed temperature was increased. At a stress temperature of 60° C., the acidic variants were baseline separated from the main peak with a pI of 7.4 (FIG. 6). This suggests that the higher the stress temperature, the greater the “acidic variant”.

The LNP stability experiment described above was repeated using LNPs that were formulated without mRNA. The corresponding electropherograms shown in FIG. 7 had a different degredation pattern compared to the mRNA containing LNPs described above (and shown in FIG. 6). At 45° C. after one day, the entire empty LNP peak showed an acidic shift of approximately 0.1 pI units. A sample stressed at 60° C. for one day showed an uncharacteristic peak profile containing a sharp spike in absorbance, which may indicate LNP destabilization or aggregation. Unlike the mRNA containing LNPs, these preparations did not show splitting into two peaks or generation of “acidic variants”, indicating that the previously seen acidic peak may be due to the mRNA being exposed on the surface of the LNP.

### REFERENCES (EACH OF WHICH IS HEREBY INCORPORATED BY REFERENCE IN ITS ENTIRETY)

1. Abrams, M. T., et al.; Mol Ther 2010, 18, 171-180.
2. Adams, D., et al.; New England Journal of Medicine 2018, 379, 11-21.
3. Allen, T. M., Cullis, P. R.; Science 2004, 303, 1818-1822.
4. Anderson, C. L., Wang, Y., Rustandi, R. R.; Electrophoresis 2012, 33, 1538-1544.
5. Arnaud, I., Abid, J. P., Roussel, C., Girault, H. H.; Chem Commun (Camb) 2005, 787-788.
6. Bo, T., Pawliszyn, J.; Electrophoresis 2006, 27, 852-858.
7. Brito, L. A., et al.; Mol Ther 2014, 22, 2118-2129.
8. FDA, Liposome Drug Products Chemistry, Manufacturing, and Controls; Human Pharmacokinetics and Bioavailability; and Labeling Documentation; Guidance for Industry. Office of Communications, Division of Drug Information, Center for Drug Evaluation and Research; Food and Drug Administration 2018.
9. Foged, C. Curr. Top. Med. Chem. 2012, 12 (2), 97-107.
10. Gindy, M. E., et al., Mol. Pharm. 2014, 11, 4143-4153.
11. Gole, A. M., et al., J. Chrom. A 1999, 848, 485-490; and Arnaud, I., et al., Chem. Comm. 2005, 787-788.
12. Guan, S., Rosenecker, J., Gene Ther 2017, 24, 133-143.
13. Jeffs, L. B., et al., Pharm. Res. 2005, 22, 362-372.
14. Kedmi, R., Ben-Arie, N., Peer, D., Biomaterials 2010, 31, 6867-6875.
15. Kou, L., Sun, J., Zhai, Y., He, Z., Asian Journal of Pharmaceutical Sciences 2013, 8, 1-10.
16. Lautier, D., et al., Mol. Cell. Biochem. 1993, 122, 171-193.
17. Lin, P.J.C., et al., Nanomedicine:Nanotech. Biol. Med. 2013, 9, 233-246
18. Loughney, J. W., Ha, S., Rustandi, R. R., Anal Biochem 2017, 534, 19-23.
19. Mayer, L. D., Bally, M. B., Loughrey, H., Masin, D., Cullis, P. R., Cancer Research 1990, 50, 575.
20. Midoux, P., et al., Expert Rev.Vaccine, 2015, 14:2, 221-234.
21. Mizrahy, S., Hazan-Halevy, I., Landesman-Milo, D., Ng, B. D., Peer, D., Front Immunol 2017, 8, 69.
22. Owen, R. L., et al., Electrophoresis 2005, 26, 735-751.
23. Riley, K. R., et al., J. Chrom A 2016, 1463, 169-175.
24. Rodriguez, M. A., Armstrong, D. W., J Chromatogr B Analyt Technol Biomed Life Sci 2004, 800, 7-25.
25. Rodriguez-Diaz, R. et al., Electrophoresis 1997, 18, 2134-2144
26. Rustandi, R. et al., Humana Press, New Jersey, 2013, vol. 988, pp. 181-197
27. Rustandi, R. et al.; Electrophoresis 2012, 33, 1538-1544
28. Rustandi, R. et al.; Electrophoresis 2014, 35, 1072-1078.
29. Ueda, K., Ann. Rev. Biochem. 1985, 54, 73-100.
30. van Tricht, E., et al.; Talanta 2015, 144, 1030-1035.
31. Wang, R. R., et al., Electrophoresis 2014, 35, 1072-1078
32. Wiedmer, S. K., et al., Electrophoresis 2001, 22, 1305-1313
33. Wu, J., Li, S. C., Watson, A., J Chromatogr A 1998, 817, 163-171.
34. Zatsepin, T. S., et al., I. J. Nanomed. 2016, 11, 3077-3086.
35. Zhang et al., Mol. Pharm. 2013, 10, 397-405
36. Zhang, J., Haas, R. M., Leone, A. M., Anal Chem 2012, 84, 6088-6096.

What is claimed is:

1. A method of separating lipid nanoparticles (LNPs) according to their isoelectic points, the method comprising applying a separating voltage to a separation matrix comprising carrier ampholytes and the LNPs for a sufficient time to separate the LNPs according to their isoelectic points.

2. The method of claim 1, wherein the separation matrix further comprises a stabilizer.

3. The method of claim 2, wherein the stabilizer is glycerol.

4. The method of claim 2, wherein the stabilizer is present in the separation matrix at 5-15% (w/v or v/v).

5. The method of claim 1, wherein the separation matrix further comprises methylcellulose.

6. The method of claim 5, wherein the methylcellulose is present in the separation matrix at 0.2%-0.25% w/v.

7. The method of claim 1, wherein the carrier ampholytes form a linear pH gradient when the voltage is applied.

8. The method of claim 1, wherein the carrier ampholytes form a sigmoidal pH gradient capable of higher resolution separation in a pH sub-range that includes the isoelectric points of the LNPs when the voltage is applied.

9. The method of claim 1, wherein the separation matrix is in a capillary.

10. The method of claim 9, wherein the capillary is a silica capillary coated with fluorocarbon.

11. The method of claim 1, wherein the LNPs comprise, individually, one or more cationic lipid species, one or more non-cationic lipid species, cholesterol, one or more PEG-lipids, or a combination thereof.

12. The method of claim 1, wherein the LNPs comprise, individually, one or more encapsulated nucleic acids.

13. The method of claim 1, wherein the LNPs comprise, individually, one or more encapsulated mRNA species.

14. The method of claim 1, further comprising imaging the separation matrix after the sufficient time to produce an electropherogram.

15. The method of claim 14, wherein imaging the separation matrix comprises detecting UV absorbance at 280 nm.

16. The method of claim 14, further comprising measuring a peak area corresponding to LNPs having a selected pI in the electropherogram and comparing the peak area to a calibration curve to calculate a total lipids concentration.

17. The method of claim 14, further comprising comparing the electropherogram to a reference electropherogram produced under identical conditions.

18. The method of claim 17, wherein the reference electropherogram was produced from the same batch of LNPs as the LNPs in the electropherogram, and wherein an acidic shift or altered peaks in the electropherogram indicate a change in LNP stability.

19. The method of claim 17, wherein the reference electropherogram was produced from a reference batch of LNPs having the same lipid composition as the LNPs in the electropherogram, and wherein a significant difference between the electropherograms indicates a manufacturing problem for the LNPs.

20. The method of claim 1, wherein the LNPs in the separation matrix comprise a level of cationic lipids that is normalized to a level of cationic lipids used to produce the reference electropherogram.
